(19) United States
(12) Patent Application Publication
(10) Pub. No.: US 2010/0070043 A1
(43) Pub. Date: Mar. 18, 2010

(54) SHAPE MEMORY ORTHOPEDIC JOINT

(76) Inventor: Michael S. Kitchen, Charleston, SC (US)

Correspondence Address: B. CRAIG KILLOUGH, P. O. DRAWER H, CHARLESTON, SC, 29402, US

(21) Appl. No.: 12/256,730

(22) Filed: Oct. 23, 2008

Related U.S. Application Data
(63) Continuation-in-part of application No. 11/196,891, filed on Aug. 4, 2005, now Pat. No. 7,582,109.
(60) Provisional application No. 60/982,210, filed on Oct. 24, 2007.

(51) Int. Cl. A61F 2/30

(52) U.S. Cl. 623/18.11

(57) ABSTRACT

Shape memory material to effect construction of an artificial joint. Placement is effected through a minimally invasive surgical technique, wherein the device transforms from a collapsed indeterminate form to a determinate form as heat is introduced. The device self deploys to a joint having determinate geometric shape and super elastic properties.

This Application is a continuation in part of application Ser. No. 11/196,891 filed Aug. 4, 2005.

Applicant claims the benefit of provisional application Ser. No. 60/982,210 filed Oct. 24, 2007.

### FIELD OF THE INVENTION

The invention relates to joint replacement devices and methods.

### BACKGROUND OF THE INVENTION

Degenerative joint disease of the small joints of the hands and feet affects millions of Americans. Currently available technologies are available which replace severely damaged joints with prosthetic joints; however, these techniques require open excision of the diseased joint and open placement of the prosthetic joint.

Many current designs for small joint prosthesis are based upon articulating designs that are similar to designs used for large joints. These large joint technologies are more than thirty years old. While bearing surface articulating joints are certainly expected to be highly durable, cost of manufacture is relatively high and requires open procedure type techniques to place. The articulating surface type joints closely mimic the structure of a native joint; with articulating surfaces that are formed along a radius of motion much like a native joint. In this sense, the articulating prosthetic joints are the closest thing to a natural joint allowing for natural motion between proximal and distal bones with the prosthetic joint between. This has the benefit of preserving the spatial relationships of the native ligaments through normal range of motion. Surgeries for placement of an articulating type joint in the hand are very similar to a total knee replacement procedure, while these surgeries do not typically take as long as a total knee surgery, they are lengthy.

Some prosthetic joint devices are based upon resilient polymer insertions that allow movement across a joint through plastic deformation. Resilient polymer type prosthetic joints have been available for more than twenty years; these joints permit motion with a hinge type structure that typically moves the center of motion to a volar geometry, affecting normal ligamentous relationship across the joint. Placement requires an open technique with careful dissection to preserve native collateral ligament structures to attain a good post surgical result. Of necessity these surgeries have been lengthy and are typically limited to replacement of two or three joints at a time. Longevity of soft joint replacements are dependent upon loads that they are subjected to, this technology may not be suitable for patients who use their hands even for relatively light manual labor.

There is a need for a small joint replacement device that can be placed utilizing a percutaneous technique.

There is a need for a small joint replacement device that can be manufactured for low cost.

There is a need for small joint replacement technologies that may be affected in a short operative time with multiple joints replaced at a single surgery.

There is a need for small joint replacement technologies that are composed of materials of a highly durable nature.

### SUMMARY OF THE INVENTION

The invention exploits shape memory material to effect construction of an artificial joint. Placement is effected through a minimally invasive surgical technique, wherein the device transforms from a collapsed indeterminate form to a determinate form as heat is introduced. The device self deploys to a determinate geometric shape having super elastic properties.

### DESCRIPTION OF DRAWINGS OF PREFERRED EMBODIMENTS

FIG. 1A demonstrates a shape memory joint in process of deployment. an inner component in process of deployment.

FIG. 1B demonstrates an inner component of the shape memory joint in process of deployment in process of deployment, transforming from indeterminate shape to determinate shape.

FIG. 2A demonstrates the formation of an osteotomy and an arthrotomy with an osteotomy/arthrotomy forming instrument in place.

FIG. 2B demonstrates a determinate shape memory joint in place in a first bone and a second bone, and forming a joint between the bones.

FIG. 3 is an isolation of the determinate shape memory joint demonstrating a “flower petal” geometry of selectively attenuated structure that forms a flexible section between opposite anchors, and demonstrating preferential planes of motion.

FIG. 4 shows the shape memory joint of FIG. 3 in position in a first bone and second bone by positioning in an osteotomy/arthrotomy.

FIGS. 5A, 5B and 5C demonstrate bending portion of an inner component and showing movement in flexion and extension with distortion of a flexible section between opposite anchors part of device

FIG. 6A demonstrates sequential deployment of an inner component as it transforms from indeterminate shape to determinate form within a determinate outer component sheath.

FIG. 6B demonstrates the determinate form of the deployed and positioned joint.

FIG. 7 indicates three separate portions of an embodiment of the invention, with a distal anchor 304, a flexible section 305 and a proximal anchor 306.

FIG. 8 is the device of FIG. 7 enlarged to emphasize an attenuated structure of a middle flexible section.

FIG. 9 demonstrates movement from axial loading with joint space separation at placement of the flexible section.

FIG. 10 is a cross section showing a three member flexible section inner component, with dorsal paired members configured to resist lateral movement at the joint.

FIGS. 11A-D demonstrate various embodiments of an attenuated flexible section.

FIG. 12 demonstrates compression and extension allowance at the flexible section.

FIGS. 13A-C demonstrate relative movement of the first bone to the second bone with an embodiment of the joint device in position.

FIGS. 14A-C demonstrate relative movement of the first bone to the second bone with an embodiment of the joint device in position.

FIGS. 15A-C show the device as a phantom and in position, with fastener embodiments demonstrated for holding the anchors.

### DESCRIPTIONS OF PREFERRED EMBODIMENTS

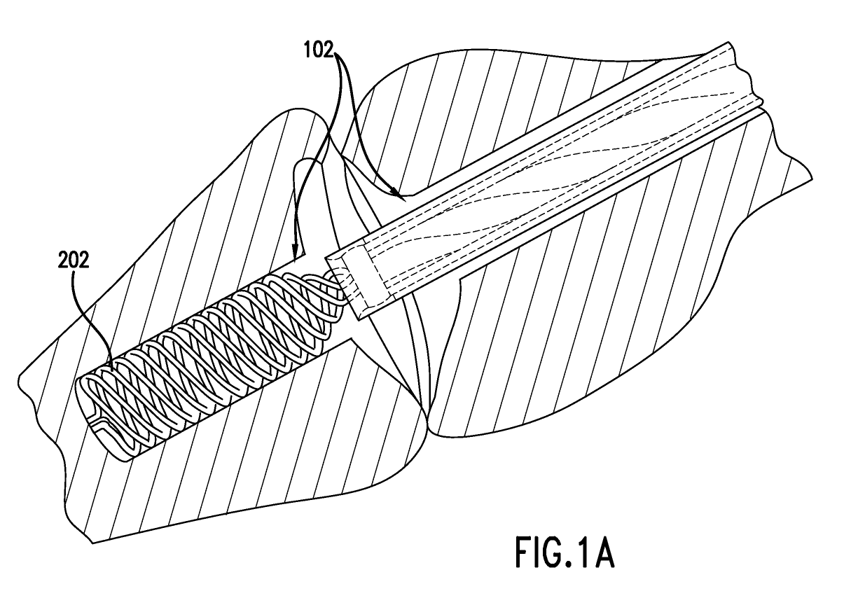

In one embodiment, the device comprises first and second shape memory component. Each component may be separately placed into a surgically formed osteotomy/arthrotomy 102 utilizing a thermal transition method of deployment across a native joint space. FIG. 1. The thermal method of deployment is described in U.S. Patent Application Publication No. 2006/0030933, which is incorporated herein by reference. The transitioned final form of the joint is realized in situ and is capable of adopting a myriad of complex three dimensional forms. This method effects an ordered, sequential and predictable introduction of heat to a thermally active shape memory material in substantially linear form.

The first placed component in this embodiment may be an anchor 202 which is introduced into the preformed osteotomy/arthrotomy utilizing the thermal method of deployment. The anchor may form an outer sheath that is coiled, or helical. The anchor holds the device in the osteotomy in the associated bone. A second placed component, which may continuously formed with the anchor, is a flexible section 206, which acts as a hinge. The anchor is sized at deployment to allow tolerance so that the device may be rotated within the osteotomy/arthrotomy post deployment. Radial positioning of the component allows bending at the flexible section in a biased manner, which allows for bending preferentially in a single plane or multiple planes with greater resistance to bending in other planes. Positioning movement allows the device to be positioned along the long axis of the osteotomy/arthrotomy, such that the portion of the component with greatest diameter, section 206, shown as having a flower petal configuration, may be placed between the bones to coincide with the portion of the arthrotomy 104 having the greatest diameter.

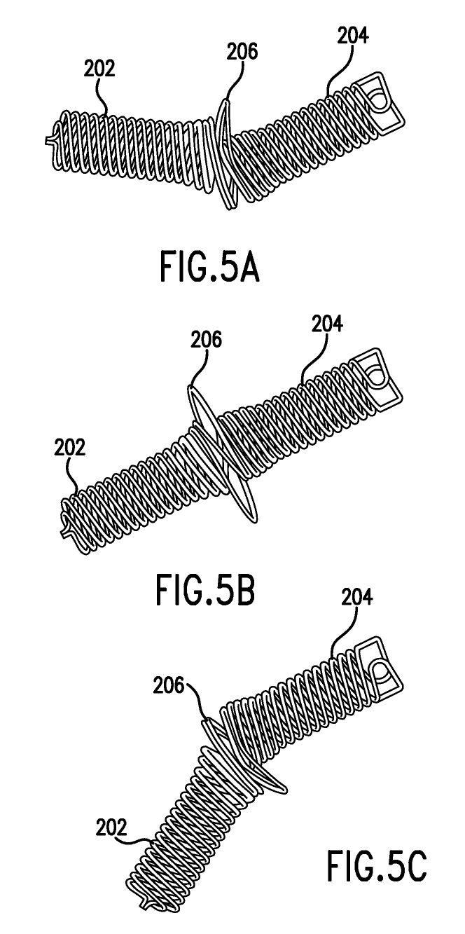

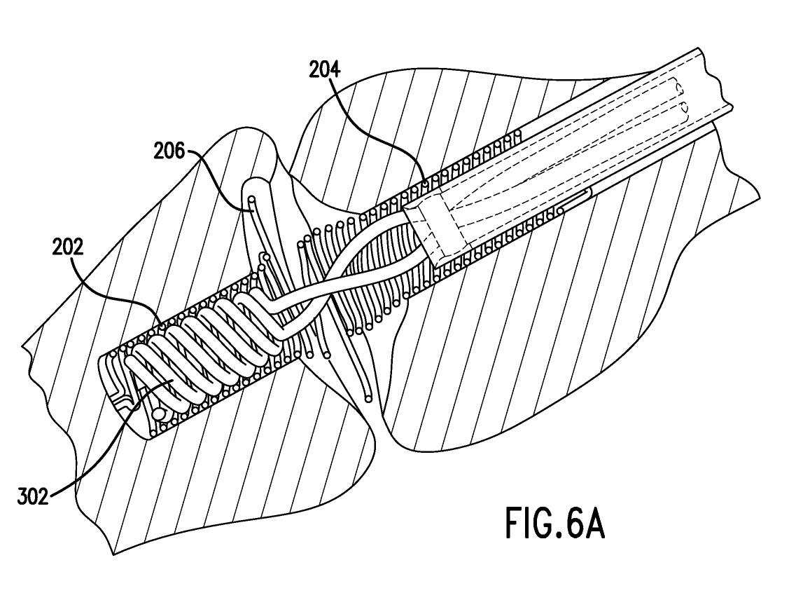

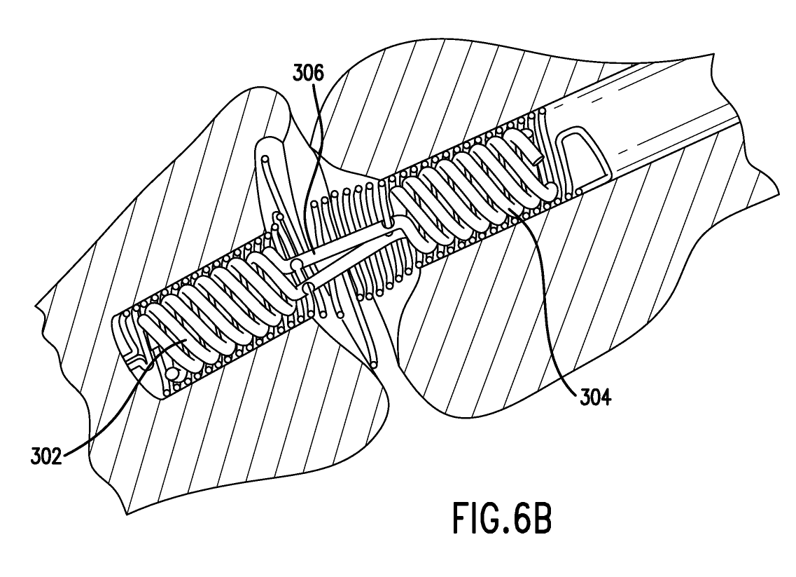

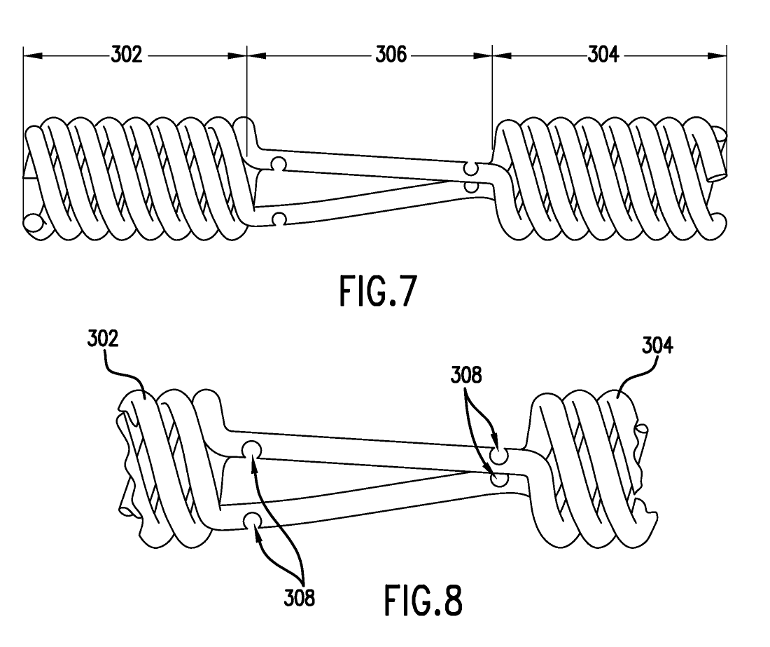

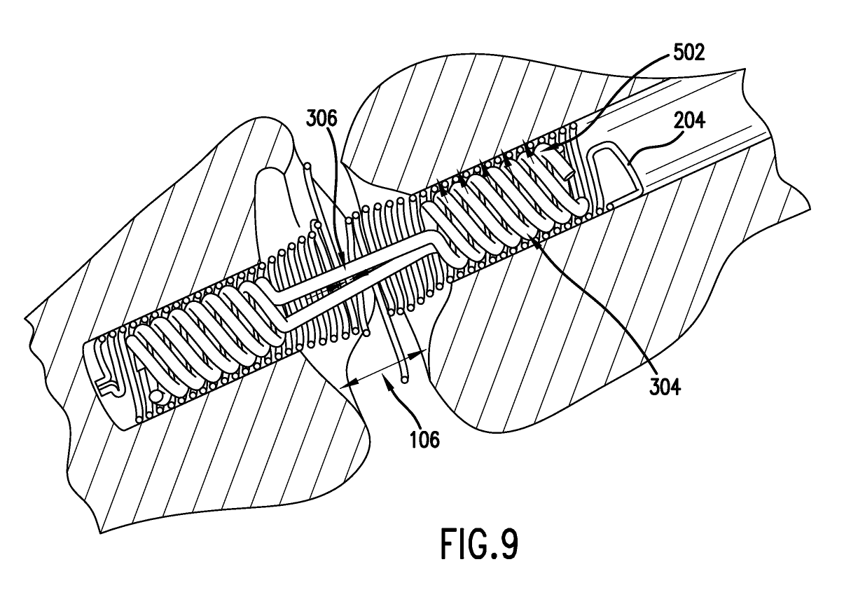

The flexible section in this embodiment is in an outer sheath, with helical wires expanded outwardly in what may be a “flower petal” like arrangement. This section may be formed of one or more windings, and has a diameter that is greater than the diameter of the osteotomy. This enlarged diameter accommodates bending at this portion that is positioned between the bones, thereby forming a flexible joint between anchor 202 and anchor 204. The enlarged winding or “flower petal” geometry creates a section with attenuated structural properties, such that the distal cylindrical portion may move through a predetermined radius relative to the proximal cylindrical portion. FIG. 5. The length of the radius through which the two cylindrical portions move relative to one another may be determined by the length of the “bending arm” of the inner hinge component.

A second or inner component may be placed utilizing the thermal method of deployment. The inner component may be placed concentrically within the outer component or outer sheath. FIG. 1B. The act of deployment of the inner component serves to lock the entire construct in a rigid manner: the outside diameter of the inner component may be slightly larger than the inner diameter of the outer sheath, thus upon deployment the outer component anchors are expanded within the osteotomy/arthrotomy locking the anchors into place. The portions of the outer component anchors may be locked into place sequentially: i.e. the distal portion 302, then the middle portion is placed under axial compressive load opening the joint space 106, and finally as the inner component is deployed in the area coincident with the outer component proximal anchor 204, this portion is locked into position within the osteotomy/arthrotomy.

Geometrically, the inner component may be comprised of three separate sections from distal to proximal: an inner or distal portion 302, which may be cylindrical, that expands within the distal anchor 202, a flexible section, which may be a middle “bending arm” section having attenuated structure 306 that favors bending in a specific plane or planes, and functions to separate under stress at placement the distal and proximal portions of the outer component, thus opening the joint space 106, and the proximal portion 304 that expands at deployment, locking the proximal anchor 204 of the outer component into the formed osteotomy/arthrotomy 502.

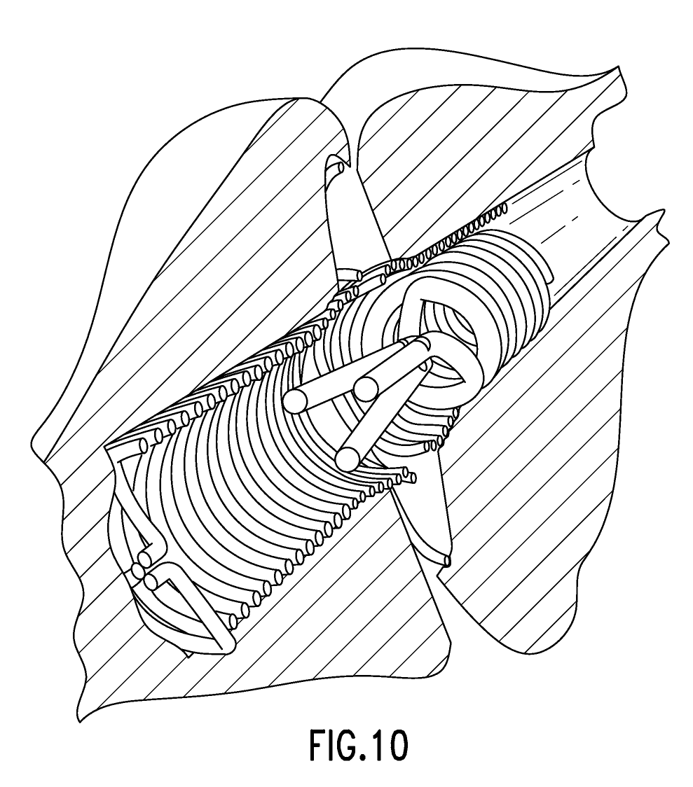

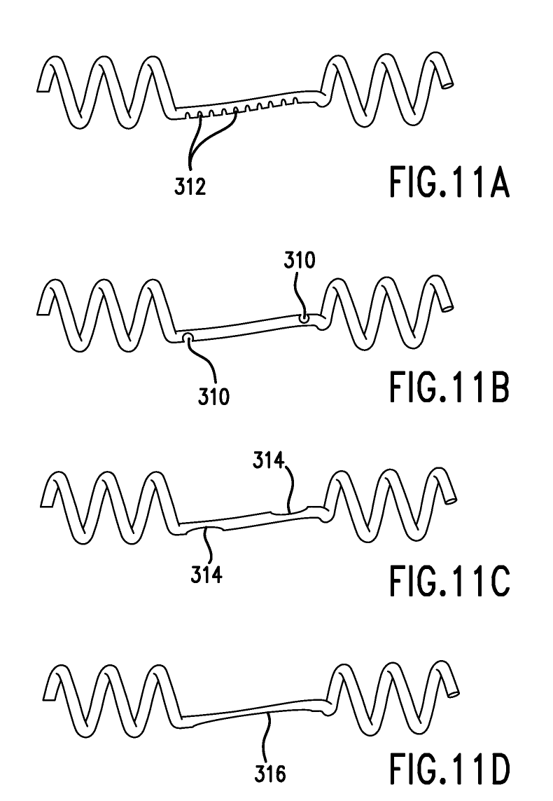

The inner component of this embodiment may by designed at the flexible middle section to favor motion in a specified plane or planes, which may be accomplished through selective attenuation 308 of flexible middle section 306. Further, this selective attenuation allows the structure to resist bending in a plane or planes that would be unfavorable. In one embodiment, the flexible “bending arm” section has three round wire members that are so arranged that there is a single member at the most volar aspect and two wires that are at the most dorsal aspect. FIG. 10. This embodiment provides a structure that is capable of resisting lateral bending through this portion of the device. Structural attenuation is configured to reduce the structural cross section of each member in the volar/dorsal plane which is the natural plane of flexion and extension for a finger of a human into which the device is installed. The device may have structural attenuation at the distal and proximal portions of the flexible “bending arm” section 306; however, multiple structural attenuations may be made through the entire “bending arm” portion of the device as required to accommodate motion in any specified plane or multiple specified planes. Four exemplary designs to achieve structural attenuation are shown including: 1) multiple symmetric cuts, which may be laser cut, placed along a length of flexible section or bending arm 312; 2) single cuts placed proximally and distally on opposing sides at bending arm 310 to accommodate bending at defined areas; 3) large proximal and distal cuts providing a longer length of attenuated cross section of the bending arm 314; and 4) attenuation by reduced dimension along a substantial portion of the length of the bending arm 316.

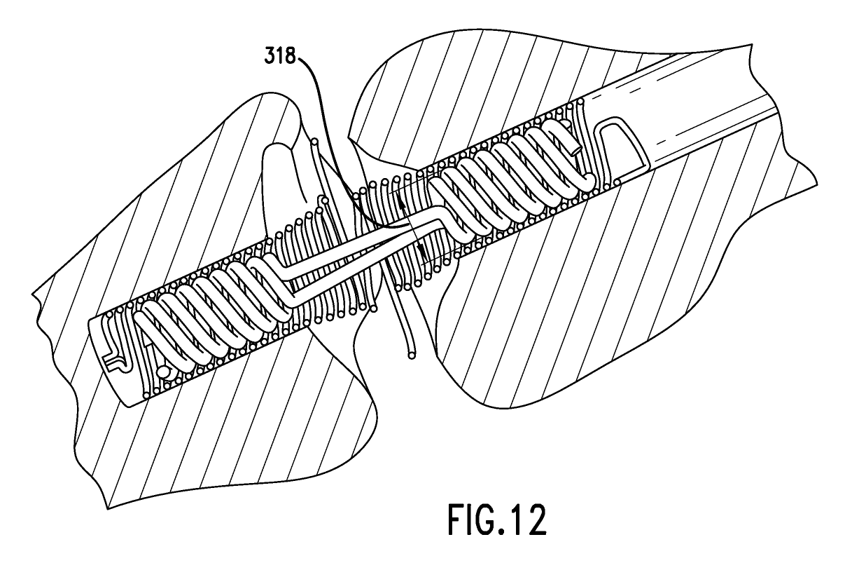

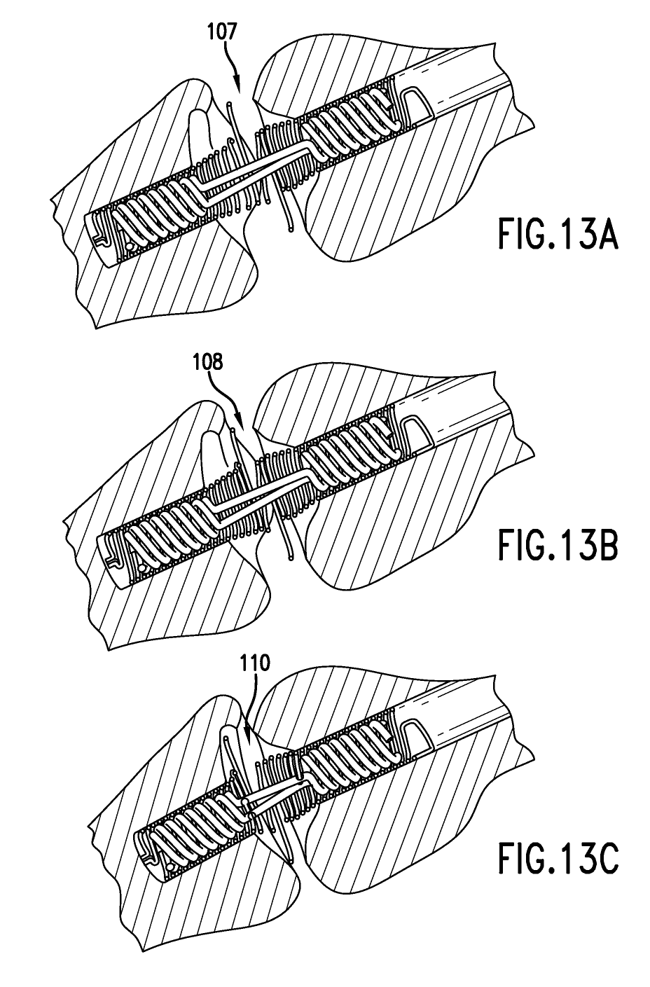

Mechanical relationships may be maintained between the geometrically distinct areas of the device through flexion and extension motions. The “bending arm” portion of the inner component serves to maintain a specified distance between distal and proximal cylindrical portions of the device while favoring bending in a specified plane or planes. The “bending arm” portion of the inner component may be configured to allow for a controlled degree of compression or axial expansion along its length by having short segments of helix that are of less diameter than required to engage the anchor. See 318 of FIG. 12. This characteristic allows for imprecise placement of the prosthesis relative to the natural center of motion of the native joint.

It is anticipated that the device may be secured within the osteotomy/arthrotomy though expansive force applied to the device as the inner component is deployed. However, there are alternate means through which it is anticipated that the device may be secured in place. The device may be secured utilizing small diameter screws 500 placed axially or through a side wall. The device may be secured utilizing bone staples 504 placed axially or through a side wall. The device may be coated with bone growth or stimulation factors to promote fusion at points of contact. The device may be filled with native bone graft or artificial bone graft 506 to promote fusion. The device may be secured utilizing artificial or naturally derived adhesives capable of bonding to bone.

Functional relationships of the device to native bone and joint structures are designed to closely mimic natural function of the joint while alleviating common pathologic conditions that affect the joints of the human body. Placement technique utilizes an arthrotomy procedure in addition to an osteotomy; this component of the procedure serves to remove pathologic joint tissue and place the articulating joint surfaces at a distance from each other thus reducing chronic change processes that result from “bone on bone” conditions of osteoarthritis and rheumatoid disease processes. The joint is designed to provide a radius of rotation with the distal cylindrical portion moving along an arc of predetermined distance from the proximal cylindrical portion; the structural relationship forming this linkage may be rigid in nature with an absolute fixed distance between the two cylindrical portions or the relationship may be semi-rigid allowing for some extension and/or compression along the length of the “bending arm”. The joint will separate with tension applied along its axis 107 as compared with its neutral position 108. Compressive force applied along the axis of the joint will cause the joint space to diminish 110. Compressive and extensile motion is accommodated by providing a short segment of free helix at the proximal and/or distal most part of the flexible “bending arm” section, said free helix having a controlled resistance to force applied along the axis of the flexible “bending arm” section 306.

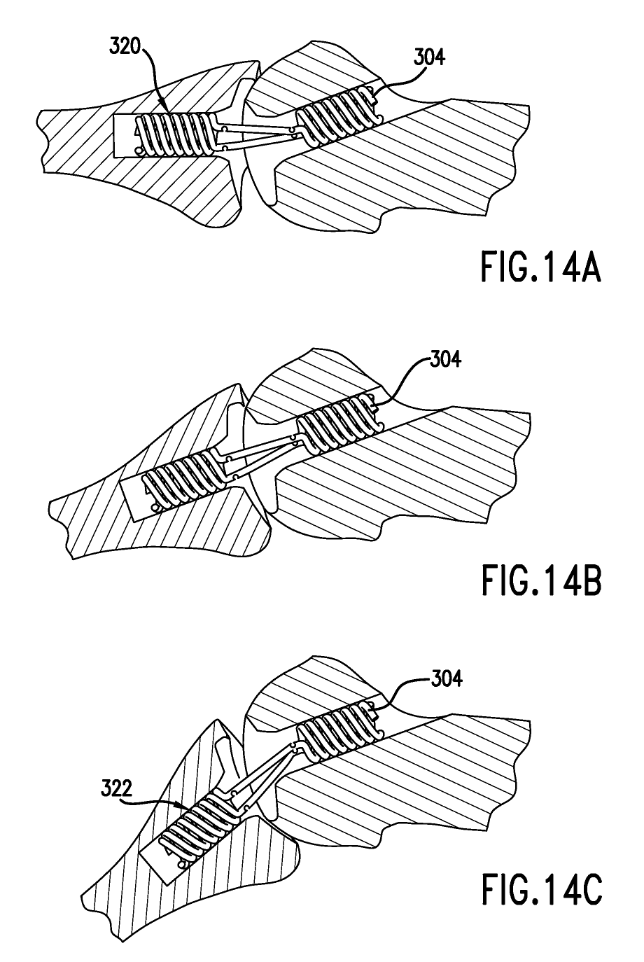

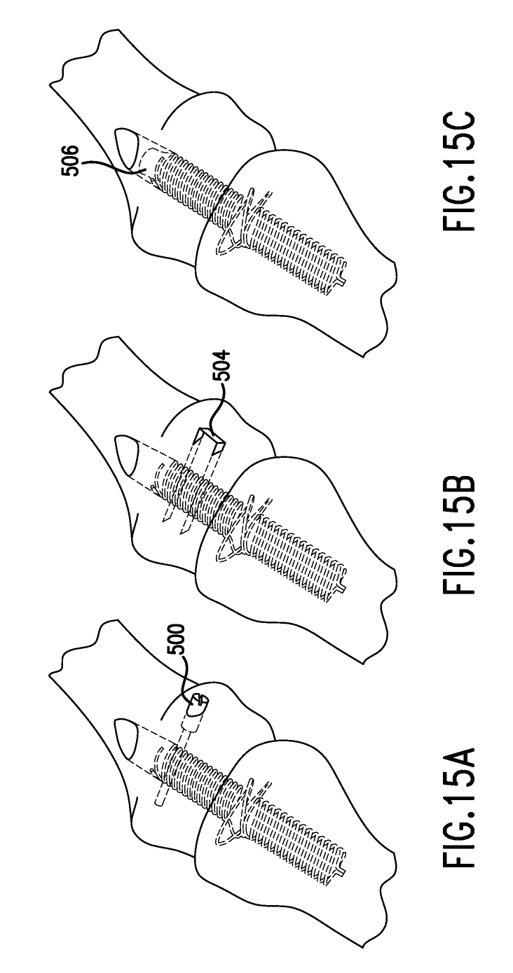

In another embodiment, the outer component or outer sheath is omitted. The inner component of the device as described herein serves forms the prosthetic joint. FIG. 14. Lateral stability as well as bending motion is accomplished with the bending arm portion of the device. Similar to the multi-layered embodiment of the device described in the preferred embodiment description the device is placed utilizing the thermal method of deployment into a preformed osteotomy/arthrotomy. This embodiment allows for extension 320 and flexion 322 as with the two component system described herein.

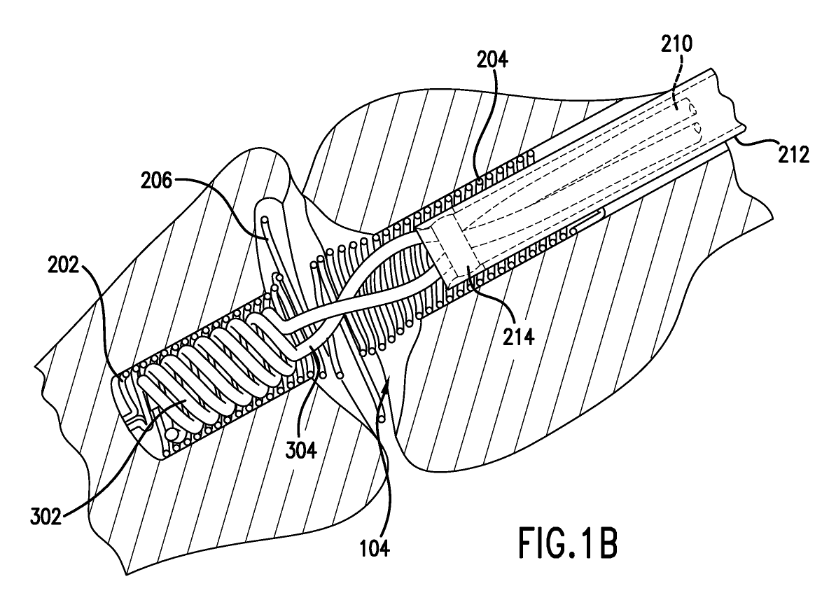

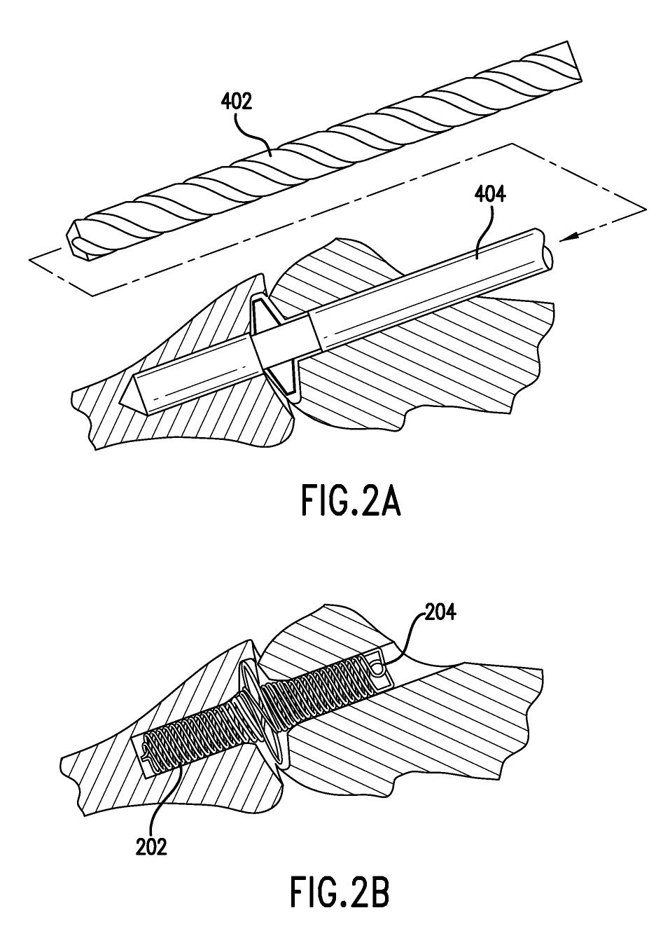

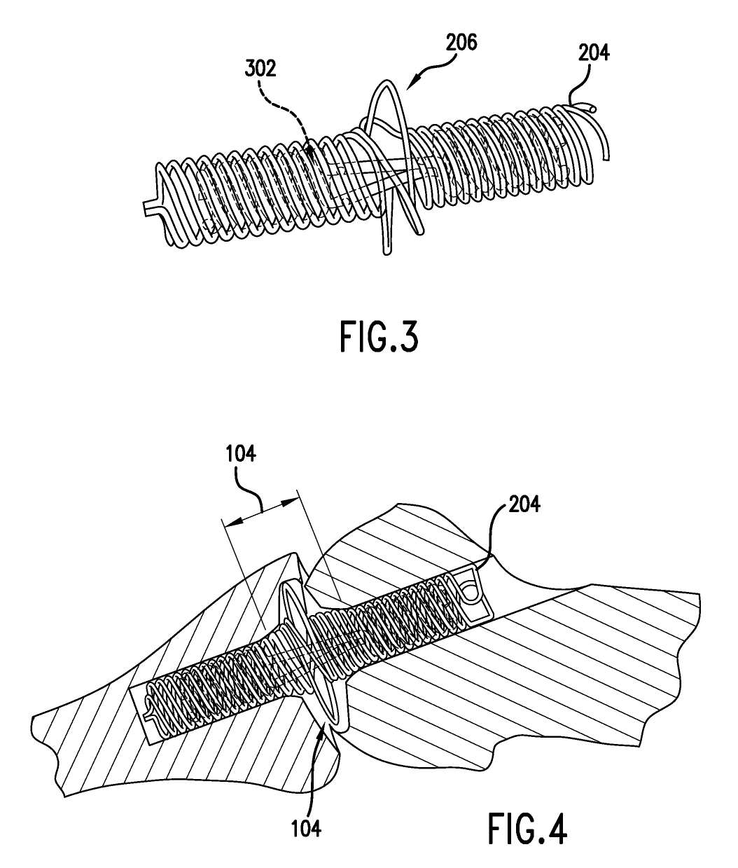

The device as positioned for use in the spine is preferred to be a coiled structure, and may be helical in structure. Exemplary coiled structures are shown in FIG. 2. The device may be formed as a level cylinder 22 (FIG. 2 A), a conic cylinder 24 (FIG. 2 B), a lozenge shape 26 (FIG. 2 C), and configured with a rectangular cross section 28 (FIG. 2 D). As long as the final form is maintained at temperatures above the design transition temperature, the device will remain in its super-elastic austenite “shape memory” or determinate form with high strength structural capabilities.

The device is constructed to maintain a super-elastic form at body temperature, and may assume this shape at slightly below body temperature. This final deployed form of the device has a shape and size that may be a coiled or helical structure at opposite ends, with the anchoring ends joined by flexible section or arm. The structural properties of the device, when maintained at temperatures at or above the transition temperature, which is preferred to be at, or slightly below, body temperature is able to correct or assist in correcting joint dysfunction between adjoining bones.

Currently available materials meeting desirable specifications for formation of the shape memory joint are various alloys of nitinol or nitinol like alloys. Alloy composition may be adjusted, creating shape memory materials having super-elastic and shape set characteristics (austenite state) near body temperature, while retaining those shape characteristics at body temperature and higher temperatures. These alloys exist at lower temperatures in martensite state wherein the material is relatively malleable and has no shape set or super-elastic properties, the shape may be expressed as “indeterminate” at these temperatures. When the shape of the device is indeterminate, if a dynamic force is placed upon the device and the dynamic force changes the shape, the shape into which the device is changed is retained when the dynamic force is removed. This martensite state corresponds to the pre-deployment malleable form, or indeterminate form, of the device. In this state, the device may be linear, like a wire, and may be bent or shaped like a wire. In a preferred embodiment, the wire 210 may be shaped manually by a physician installing the device. FIG. 1.

When heat is applied to the device, the device assumes its predetermined super-elastic austenite “shape memory” form with high strength and predicable shape and structural capabilities. The device will retain this shape as long as the temperature is maintained above the predetermined temperature, which is preferred to be just below body temperature of the human or other vertebrate into which the device is to be positioned. When a dynamic force is not being actively applied to the device at this higher temperature, the device assumes and retains a predetermined shape, which may be summarily referred to as a determinate shape. The device is shown in various embodiments of determinate shape in the drawing Figures.

In one embodiment, the device is a wire 210 having a substantially round cross section. The determinate form of the device is shape set to a coiled or helical form for anchors 202 and 204 and ends 302 and 304. FIGS. 1A, 1B. The wire is maintained at a temperature below M_{f} (martensite final state) within the deployment catheter prior to placement. At this temperature, the wire is readily formable with little force required to bend or shape the wire, and the wire may be pushed through a lumen of a flexible tube 212. The tip of a catheter may be introduced to the depth of the cylindrical osteotomy. Heat is then introduced at the catheter tip, such as by the use of electrical resistance coils 214, transitioning the shape of the memory material to its determinate shape as it is exposed to heat and as it exits the tip of the catheter. The temperature environment proximal to the catheter tip is maintained below M_{f}. Temperatures after the catheter tip are maintained at greater than A_{f} (body temperature or slightly below). Transition to austenite form proceeds linearly along the length of the device in an antigrade fashion: distal to proximal.

The device may be repositioned during the placement process by terminating heat introduction, and pulling the device in the opposite direction and into the catheter, where the temperature environment is less than M_{f}. Stated otherwise, the transition process is reversed.

The device is placed into an osteotomy formed by a tool 402 in the adjoining bones. A tool 404 may also be used to form an arthrotomy. FIG. 2A; FIG. 2B. The device is positioned with an anchor 202, 204 in each of the adjoining bones, with the flexible section between the bones in the arthrotomy, forming a flexible joint. The flexible section may be formed of windings having a dimension that is larger than the anchors and the osteotomy, such as windings that extend beyond the diameters of the anchors and the osteotomy.

What is claimed is:

1. A thermally active device for implantation as a therapeutic joint, comprising:
a thermally active member having indeterminate shape below a transition temperature, and
above said transition temperature, said thermally active member comprises a determinate first anchor and a determinate second anchor, wherein a determinate flexible section is disposed between said determinate first anchor and said determinate second anchor, wherein said determinate first anchor occupies a space within an osteotomy of a first bone, and said determinate second anchor occupies a space within an osteotomy of a second bone, and wherein said flexible section forms a flexible joint between said first bone and said second bone.

2. A thermally active device for implantation as a therapeutic joint as described in claim 1, wherein said thermally active member is encapsulated.

3. A thermally active device for implantation as a therapeutic joint as described in claim 1, wherein said thermally active member is encapsulated in a polymer.

4. A thermally active device for implantation as a therapeutic joint as described in claim 1, wherein said first anchor portion comprises a biologic fusion enhancement agent.

5. A thermally active device for implantation as a therapeutic joint as described in claim 1, wherein said thermally active member is present within a container when implanted.

6. A thermally active device for implantation as a therapeutic joint as described in claim 1, wherein said thermally active member is present within a balloon when implanted.

7. A thermally active device for implantation as a therapeutic joint as described in claim 1, wherein said thermally active member comprises a textured outer surface.

8. A thermally active device for implantation as a therapeutic joint as described in claim 1, wherein said thermally active member is attenuated at said flexible section.

9. A thermally active device for implantation as a therapeutic joint as described in claim 1, wherein said thermally active member that is below said transition temperature is capable of being translocated through an elongated lumen.

10. A thermally active device for implantation as a therapeutic joint as described in claim 1, wherein said thermally active member is capable of manual formation into an elongated substantially linear shape when said thermally active member is below said transition temperature.

11. A thermally active device for implantation as a therapeutic joint as described in claim 1, wherein said thermally active member that is below said transition temperature is capable of being translocated through an elongated lumen that is present within a flexible tube.

12. A thermally active device for implantation as a therapeutic joint as described in claim 1, wherein said thermally active member that is below said transition temperature is capable of being formed in a substantially linear shape and translocated through an elongated lumen.

13. A thermally active device for implantation as a therapeutic joint as described in claim 1, wherein said predetermined temperature is a temperature below a body temperature of an animal into which the thermally active member is implanted.

14. A thermally active device for implantation as a therapeutic joint as described in claim 1, wherein said flexible member is positioned between first bone and said second bone and said flexible member changes in shape in response to a shift of a dynamic load between said first bone and said second bone.

15. A thermally active device for implantation as a therapeutic joint as described in claim 1, wherein said flexible member is positioned between first bone and said second bone and said flexible member changes in shape in response to a shift of a dynamic load between said first bone and said second bone, and said thermally active member permits flexion and extension of one of said first bone and said second bone relative to a remaining bone of said first bone and said second bone.

16. A thermally active device for implantation as a therapeutic joint as described in claim 1, wherein said flexible member is positioned between first bone and said second bone and said flexible member changes in shape in response to a shift of a dynamic load between said first bone and said second bone, and said thermally active member permits anterior and posterior movement and flexion and extension of one of said first bone and said second bone relative to a remaining bone of said first bone and said second bone.

17. A thermally active device for implantation as a therapeutic joint as described in claim 1, wherein said flexible member comprises a winding that extends between a joint between said first bone and said second bone and said winding extends beyond a diameter of an osteotomy in said first bone.

18. A thermally active device for implantation as a therapeutic joint as described in claim 1, wherein said flexible member comprises a plurality of windings that extend between a joint between said first bone and said second bone and said winding extends beyond a diameter of an osteotomy in said first bone.

19. A thermally active device for implantation as a therapeutic joint as described in claim 1, wherein said determinate flexible section comprises a winding that extends between a joint between said first bone and said second bone and said winding extends beyond a diameter of said determinate first anchor.

20. A thermally active device for implantation as a therapeutic joint as described in claim 1, wherein said determinate flexible section flexes under normal muscular force associated with a joint of an animal into which the thermally active member is placed.

21. A thermally active device for implantation as a therapeutic joint as described in claim 1, wherein said determinate flexible section comprises a generally linear portion that connects said determinate first anchor to said determinate second anchor.

22. A thermally active device for implantation as a therapeutic joint as described in claim 1, wherein said determinate first anchor comprises a coiled section.

23. A thermally active device for implantation as a therapeutic joint as described in claim 1, wherein said determinate flexible section flexes under normal muscular force associated with a joint of an animal into which the thermally active member is placed, and wherein said determinate first anchor comprises a coiled section and wherein said determinate second anchor comprises a coiled section.

24. A thermally active device for implantation as a therapeutic joint as described in claim 1, wherein said determinate first anchor comprises an outer portion that surrounds an inner section.

25. A thermally active device for implantation as a therapeutic joint as described in claim 1, wherein said determinate first anchor comprises a coiled outer portion that surrounds a coiled inner section.

26. A thermally active device for implantation as a therapeutic joint as described in claim 1, wherein said determinate first anchor comprises a coiled outer portion that surrounds a first coiled inner section, said determinate second anchor comprises a coiled outer portion that surrounds a coiled inner section, said coiled inner section of said determinate first anchor is joined to said coiled inner section of said determinate second anchor by a substantially linear flexible section, and a flexible winding positioned between and extending beyond said coiled outer portion of said determinate first anchor and said coiled outer portion of said determinate second anchor, wherein said determinate flexible section formed by said substantially linear flexible section and said flexible winding flexes under normal muscular force associated with a joint of an animal into which the thermally active member is placed.
